(19) United States
(12) Patent Application Publication
(10) Pub. No.: US 2026/0193542 A1
(43) Pub. Date: Jul. 9, 2026

(54) OFFSHORE PLATFORM SYNGAS CONVERSION

(71) Applicant: BASF Corporation, Florham Park, NJ (US)

(72) Inventors: Paul LEWANDOWSKI, Freeport, TX (US); Amit A. GOKHALE, Houston, TX (US); Irina YARULINA, Winterhur (CH); Dina ZAKGEYM, Ludwigshafen am Rhein (DE); Hong LU, Houston, TX (US)

(21) Appl. No.: 19/135,224

(22) PCT Filed: Dec. 11, 2023
(86) PCT No.: PCT/US2023/083371

§ 371 Date: Jun. 3, 2025

Related U.S. Application Data
(60) Provisional application No. 63/432,335, filed on Dec. 13, 2022.
(60) Provisional application No. 63/447,700, filed on Feb. 23, 2023.

(51) Int. Cl. C10G 2/00; C07C 41/09; C07C 43/04

(52) U.S. Cl. CPC C10G 2/33; C07C 41/09; C07C 43/043; C10G 2/341; C10G 2/343

(57) ABSTRACT

Disclosed herein are methods and systems for syngas conversion on offshore platforms. In at least one embodiment, a method for converting an offshore syngas feed into a product stream comprises: directing the syngas feed to a first catalyst bed comprising a first catalyst to produce a treated stream, the treated stream comprising dimethyl ether (DME), methanol, and unconverted hydrogen generated from contact of the syngas feed with the first catalyst bed; directing the treated stream toward a second catalyst bed comprising a mixture of the first catalyst and a second catalyst; and subsequently directing the treated stream toward a third catalyst bed comprising the second catalyst without the first catalyst to at least partially convert the treated stream into the product stream.

### CROSS-REFERENCE TO RELATED APPLICATION(S)

This application claims the benefit of priority of U.S. Provisional Patent Application No. 63/432,335, filed Dec. 13, 2022, and U.S. Provisional Patent Application No. 63/447,700, filed Feb. 23, 2023, the disclosures of which are hereby incorporated by reference herein in their entireties.

### FIELD OF THE DISCLOSURE

The present disclosure relates to conversion of dimethyl ether from syngas feeds, and, more specifically, to syngas feeds of offshore platforms.

### BACKGROUND OF THE DISCLOSURE

Hydrocarbons are essential in modern life and used as fuel and raw materials, including the chemical, petrochemical, plastics, and rubber industry. Fossil fuels, such as oil and natural gas, are composed of hydrocarbons with a specific ratio of carbon to hydrogen. Despite their wide application and high demand, fossil fuels also have limitations and disadvantages. Ecological as well as economic considerations have been the impetus for research into alternative fuels, as well as for methods to reduce the environmental impact of offshore operations.

Flaring is performed on offshore platforms to burn off waste and excess gases and is a critical process to maintain the safety of the platform, protect equipment, and avoid directly introducing hazardous gases directly into the atmosphere. About 1-5% of the total feed from oil wells are vented and flared. For offshore cases, it is especially difficult to build additional infrastructure to transport the vapor in lieu of flaring. Converting the vapor stream to liquids is valuable from a gross product perspective, and is also helpful if regulations eliminating flaring are imposed. Most existing gas-to-liquid processes require severe operating conditions which increases the difficulty of building and operating these processes on a platform. Further, existing solutions generally have difficulties reducing byproducts and maximizing high-octane products which are valuable to the gasoline stream. Moreover, existing catalysts that generate high-octane products typically form a solid byproduct of hexamethylbenzene, which is difficult to process in small units where bulk volume cannot dissolve the solid or when fouling is a great hindrance to feasibility.

### SUMMARY OF THE DISCLOSURE

The following presents a simplified summary of various aspects of the present disclosure in order to provide a basic understanding of such aspects. This summary is not an extensive overview of the disclosure. It is intended to neither identify key or critical elements of the disclosure, nor delineate any scope of the particular embodiments of the disclosure or any scope of the claims. Its sole purpose is to present some concepts of the disclosure in a simplified form as a prelude to the more detailed description that is presented later.

One aspect of the present disclosure relates to a method for converting an offshore syngas feed into a product stream comprising branched alkanes, the method comprising: directing the syngas feed to a first catalyst bed comprising a first catalyst to produce a treated stream, the treated stream comprising dimethyl ether (DME), methanol, and unconverted hydrogen generated from contact of the syngas feed with the first catalyst bed; directing the treated stream toward a second catalyst bed comprising a mixture of the first catalyst and a second catalyst; and subsequently directing the treated stream toward a third catalyst bed comprising the second catalyst without the first catalyst to at least partially convert the treated stream into the product stream.

In at least one embodiment, the conversion of the syngas feed into the product stream is performed via one pass in a single-pass reactor.

In at least one embodiment, the method further comprises: separating the product stream into a predominantly aqueous stream and a predominantly organic stream comprising light fractions solubilized in reaction products; and introducing the organic stream into a crude oil pipeline.

In at least one embodiment, the organic stream has a Research Octane Number (RON) of greater than 90 or greater than 95. In at least one embodiment, the organic stream has greater than 5 wt % of aliphatic hydrocarbons with a RON of greater than 100.

In at least one embodiment, the syngas feed is contacted with the first catalyst bed in a first reactor of a two-stage reactor. In at least one embodiment, the treated stream is contacted with the second catalyst bed and the third catalyst bed in a second reactor of the two-stage reactor.

In at least one embodiment, the reaction conditions of one or more of the first reactor or the second reactor comprise a temperature of about 250° C. to about 280° C., or about 255° C. to about 265° C., and a pressure of about 10 bar to about 40 bar, or about 15 bar to about 20 bar. In at least one embodiment, the reaction conditions further comprise a flowrate of about 0.3 to about 15 g_{DME}/g_{catalyst}/hour (e.g., about 0.5 to about 3 g_{DME}/g_{catalyst}/hour, or about 0.5 to about 1.5 g_{DME}/g_{catalyst}/hour).

In at least one embodiment, the two-stage reactor is in a parallel fixed bed configuration. In at least one embodiment, the two-stage reactor is in a moving bed configuration.

In at least one embodiment, contact of the treated stream with the second catalyst bed further converts additional methanol to DME.

In at least one embodiment, a H₂/CO ratio of the syngas feed is from about 1.5 to about 3, and the CO₂ concentration is from about 5 to about 10 mol %.

In at least one embodiment, the first catalyst comprises ZnO, CuO, alumina, and optionally ZrO₂. In at least one embodiment, the first catalyst further comprises one or more of boron oxide, niobium oxide, tantalum oxide, phosphorus oxide, or combinations thereof. In at least one embodiment, the first catalyst further comprises ceria.

In at least one embodiment, the second catalyst comprises a zeolite. In at least one embodiment, the zeolite is a copper-impregnated beta zeolite. In at least one embodiment, the copper-impregnated beta zeolite comprises copper from about 2 wt. % to about 10 wt. % based on a total weight of the copper-impregnated zeolite. In at least one embodiment, the copper-impregnated zeolite further comprises one or more additional metals from about 0.5 wt % to about 3 wt %, the one or more additional metals being selected from metals having an H₂ adsorption energy of 77 KJ/mol to 124 KJ/mol on (111), (110), or (0001) surfaces.

In at least one embodiment, a silica-to-alumina ratio (SAR) of the zeolite is from about 25 to about 100, or from about 30 to about 60.

In at least one embodiment, a total surface area of the copper-impregnated beta zeolite is from about 350 m²/g to about 450 m²/g, and a pore volume of the copper-impregnated beta zeolite is from about 0.2 cm³/g to about 0.4 cm³/g.

In at least one embodiment, an amount of sites oxidizable by N₂O on the copper-impregnated beta zeolite is from about 10 μmol/g to about 30 μmol/g.

In at least one embodiment, conversion of the treated stream into the product stream results in less than about 4 wt % hexamethylbenzene.

In at least one embodiment, the method further comprises: cooling the product stream to a temperature from about 30° C. to about 70° C.; and utilizing remaining organic vapor for steam generation and/or feeding the remaining organic vapor to a vessel where the vapor is contacted with a liquid crude oil stream at ambient pressure and at a flow rate that is at least 5 times higher than the flow rate of the product stream.

In at least one embodiment, the methanol content of the product stream is less than about 0.5 wt %, or less than about 0.05 wt %.

In at least one embodiment, the method further comprises: pretreating the second catalyst with a paraffin/olefin pretreatment stream at about 200° C. In at least one embodiment, the paraffin/olefin pretreatment stream comprises at least about 0.5 mol % propane in propylene. In at least one embodiment, the paraffin/olefin pretreatment stream comprises C3 and C4 olefins.

A further aspect of the present disclosure relates to a system for converting a syngas feed into a product stream comprising branched alkanes, the system comprising: a first reactor adapted to receive a syngas feed stream, the first reactor comprising a first catalyst adapted to produce a treated stream comprising dimethyl ether (DME) and methanol generated from contact with the syngas feed stream; and second reactor downstream from the first reactor adapted to receive the treated stream, the second reactor comprising a first bed of a mixture of a first and second catalyst and a second bed comprising a second catalyst adapted to at least convert the treated stream into the product stream.

In at least one embodiment, the system is adapted to perform any of the methods of the foregoing embodiments.

### BRIEF DESCRIPTION OF THE DRAWINGS

The present disclosure is illustrated by way of example, and not by way of limitation, in the figures of the accompanying drawings, in which:

FIG. 1 illustrates an exemplary reaction system for performing one or more of the methods described herein according to various embodiments;

FIG. 2 is a plot showing temperature versus dimethyl ether conversion for a catalyst prepared according to various embodiments;

FIG. 3 is a plot of conversion as a function of temperature at different feed rates for catalysts prepared according to various embodiments;

FIG. 4 is a plot showing methanol content in a reactor product stream at different temperatures using catalysts prepared according to various embodiments;

FIG. 5 is a plot showing C₄+C-selectivity versus catalyst temperature catalysts prepared according to various embodiments;

FIG. 6 is a plot showing C-selectivity at 99.9% dimethyl ether conversion for catalyst prepared according to various embodiments; and

FIG. 7 is a plot showing DME conversion and C-selectivity at varying reactor pressure for a catalyst prepared according to an embodiment.

### DEFINITIONS

As used herein, the singular forms “a,” “an,” and “the” include plural references unless the context clearly indicates otherwise. Thus, for example, reference to “a microsphere” includes a single microsphere as well as a mixture of two or more microspheres, and the like.

As used herein, the term “about” in connection with a measured quantity, refers to the normal variations in that measured quantity, as expected by one of ordinary skill in the art in making the measurement and exercising a level of care commensurate with the objective of measurement and the precision of the measuring equipment. In certain embodiments, the term “about” includes the recited number±10%, such that “about 10” would include from 9 to 11.

Also as used herein, the terms “synthesis gas” or “syngas” refer to a mixture of carbon monoxide, carbon dioxide, and hydrogen. Syngas can be produced from many sources, including natural gas, coal, biomass, or virtually any hydrocarbon feed store, by reaction with steam or oxygen. The formation of syngas with steam is strongly endothermic and requires high temperatures. Steam reforming of natural gas or shale gas is typically performed in tubular reactors that are heated externally. The process typically employs nickel catalysts on a special support that is resistant against the harsh process conditions. Typically, syngas with H₂/CO ratios in the range of 3 to 4 is obtained in this manner.

As used herein, the term “catalyst” or “catalyst composition” or “catalyst material” or “catalyst component” refers to a material that promotes a reaction.

As used herein, the term “zeolite” refers to a crystalline aluminosilicate with a framework based on an extensive three-dimensional network of silicon, aluminum and oxygen ions and have a substantially uniform pore distribution.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

### DETAILED DESCRIPTION

This disclosure is directed in certain embodiments to converting syngas streams into product streams comprising branched alkanes. More specifically, certain embodiments relate to first converting offshore syngas feeds into a stream comprising dimethyl ether (DME), followed by conversion into a product stream comprising branched alkanes. The resulting product stream can be separated into both aqueous and organic streams, with the organic stream being introduced into a crude oil pipeline. In at least one embodiment, a Research Octane Number (RON) of the organic stream is greater than 90, or greater than 95, while having at least 5 wt % of aliphatic hydrocarbons with RON greater than 100. Conversion of the DME-containing stream to the product stream can advantageously reduce hexamethylbenzene production (e.g., resulting in less than 2 wt % hexamethylbenzene based on a total weight of the product stream).

Certain embodiments of the present disclosure leverage a synergy between syngas to DME catalysts and copper-impregnated beta zeolite catalysts. For example, a two-stage catalyst bed configuration (with a DME catalyst in the first stage and copper-impregnated beta zeolite catalyst in the second stage) may be utilized at a temperature range of about 260-270° C. and a pressure of about 20-30 bar. The two-stage catalyst bed first converts synthetic gas (“syngas”) to DME and some byproducts of methanol, water, and carbon dioxide in the first stage. The methanol and DME are further converted in the second stage to branched alkanes yielding a liquid product with a RON of greater than 90, which can then be reincorporated into the crude oil pipeline. At these conditions, economic conversion is advantageously attained at a useful and practical space velocity in both steps for suitable offshore platform equipment.

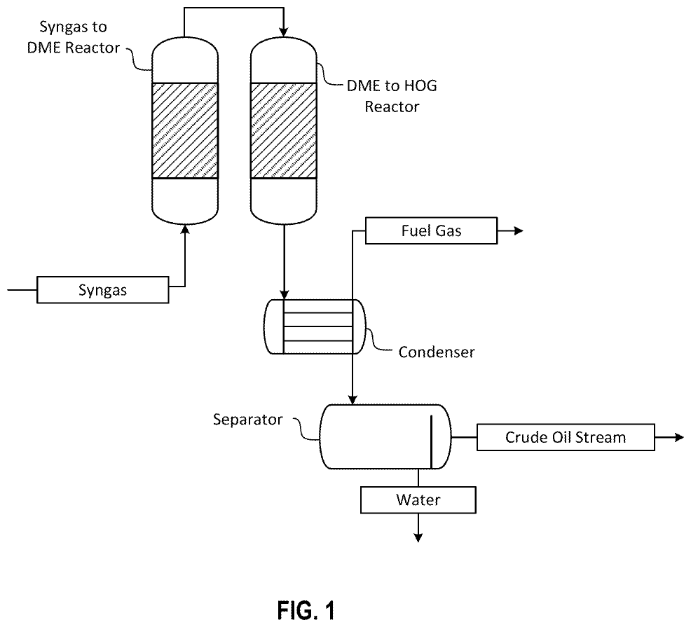

An additional benefit of these embodiments is that the amount of unconverted methanol in the reactor effluent can be minimized, reducing the oxygenates in the crude pipeline. For example, a syngas feed with a H₂/CO ratio of 1.5-2.7 can attain optimal or near optimal conversion and selectivity for a beta zeolite catalyst with unconverted H₂. The reactor effluent can then be cooled to 30-45° C. in a condenser to reduce the vapor fraction to 0.4-0.5. The aqueous stream can then be decanted and removed. The final mixed vapor/liquid organic stream can then be fed into a crude stream which will undergo a subsequent flash to separate a final flare stream. Incorporating the vapor back into the crude stream can ensure saturation of volatile C3/C4 hydrocarbons. In at least one embodiment, the zeolite catalyst is treated with a paraffin/olefin stream, preferably >1% propane in propylene, to increase catalyst activity. An exemplary two-reactor system adapted to perform this process is illustrated in FIG. 1.

### Syngas to DME Catalyst

Described now are embodiments of catalysts adapted for converting a syngas stream to a stream comprising DME. Syngas is a mixture of mainly hydrogen, carbon monoxide, and carbon dioxide, from which methanol is produced over a catalyst as follows:

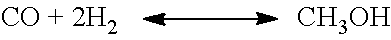

In a following step, methanol can be converted into DME by dehydration over an acidic catalyst:

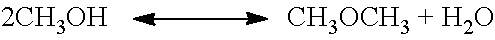

In direct DME production, there are mainly two overall reactions that occur from synthesis gas. These reactions, reaction (1) and reaction (2), are:

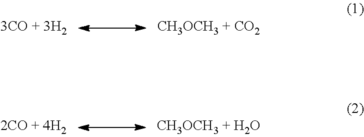

Reaction (1) occurs with the combination of three reactions, which are methanol synthesis reaction, methanol dehydration reaction, and water gas shift reaction:

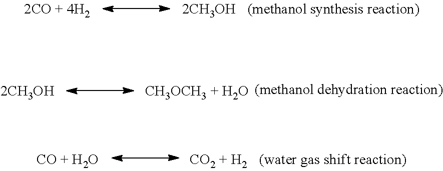

Reaction (1) has a stoichiometric ratio H₂/CO of 1:1 and has some advantages over reaction (2). For example, reaction (1) generally allows higher single pass conversions and is less energy-consuming in comparison to the removal of water from the system in reaction (2).

Suitable catalysts for the use in the syngas conversion stage can include conventionally employed methanol catalysts such as copper and/or zinc and/or chromium-based catalyst and methanol dehydration catalyst.

In at least one embodiment, a suitable catalyst may include any of those produced according to the disclosures of U.S. Pat. No. 9,295,978 B2 and U.S. Pat. No. 11,452,995 B2, the disclosure of which are hereby incorporated by reference herein in their entireties.

In at least one embodiment, a transition metal of the catalyst is selected from elements of the groups 8, 9, 10, or 11 of the periodic table, or a mixture thereof. In at least one embodiment, an amount of the transition metal(s) is about 0.01 wt % to about 20 wt %, about 0.1 wt % to about 15 wt %, or about 0.3 wt % to about 5 wt % (e.g., about 0.5 wt %) based on the total weight of the catalyst. In at least one embodiment, the transition metal is copper present in an amount from about 0.3 wt % to about 2 wt %.

In at least one embodiment, the catalyst further comprises an inorganic oxide binder material which is different from the catalytically active component. The inorganic binder material may be selected from one or more of Al₂O₃, SiO₂, TiO₂, or ZrO₂.

The amount of the inorganic oxide binder material is preferably 10 to 90 weight %, more preferably 20 to 70 weight %, specifically 30 to 50 weight %, for example 40 weight %, based on the total weight of the catalyst.

In at least one embodiment, the catalyst is formed from an acidic aluminosilicate zeolite with a SiO₂:Al₂O₃ molar ratio of from 10 to 1500:1, 50 to 1200:1, or 200 to 1000:1, for example, 280:1, 400:1, or 900:1. The catalyst may comprise, for example, about 10 wt % to about 90 wt % of at least one binder material, and about 0.01 wt % to about 20 wt % of at least one transition metal, based on the total weight of the catalyst.

In at least one embodiment, the catalyst is formed from about 30 wt % to about 80 wt % of at least one acidic aluminosilicate of framework type MFI, about 20 wt % to about 70 wt % of at least one binder material selected from Al₂O₃, SiO₂, TiO₂, ZrO₂, or a combination thereof, and about 0.1 wt % to about 5 wt % of elements of the groups 8, 9, 10, or 11 of the periodic table, or a mixture thereof, based on the total weight of the catalyst.

In at least one embodiment, a suitable catalyst for conversion of syngas to DME comprises one or more of Al₂O₃, ZnO, CuO, or ZrO₂. In at least one embodiment, the catalyst further comprises one or more of boron oxide, niobium oxide, tantalum oxide, or phosphorus oxide. In at least one embodiment, the catalyst comprises cerium oxide.

In at least one embodiment, the catalyst is in the form of particles, which may be admixed with other types of catalytic particles. In at least one embodiment, the catalyst is in the form of an extrudate.

### High-Octane Gasoline Catalyst

Described now are embodiments of catalysts adapted for converting DME to branched alkanes (e.g., triptane), referred to herein as high-octane gasoline (HOG) catalysts. In at least one embodiment, the catalyst comprises a zeolite. In at least one embodiment, the zeolite is a beta zeolite.

In at least one embodiment, the zeolite is a copper-impregnated zeolite. For example, the copper may be present from about 2 wt % to about 10 wt % (e.g., about 4 wt %) based on the total weight of the copper-impregnated zeolite.

In at least one embodiment, the copper-impregnated zeolite further comprises one or more additional metals from about 0.5 wt % to about 3 wt %. The additional metals may include palladium, nickel, ruthenium, rhenium, platinum, or combinations thereof. In at least one embodiment, the additional metals may be selected from those having an H₂ adsorption energy of 77 KJ/mol to 124 KJ/mol on (111), (110), or (0001) surfaces.

In at least one embodiment, a silica-to-alumina ratio (SAR) of the zeolite is from about 25 to about 100, or from about 30 to about 60.

In at least one embodiment, a total Brunauer, Emmett and Teller (BET) surface area of the zeolite is about 240 m²/g, about 250 m²/g, about 260 m²/g, about 270 m²/g, about 280 m²/g, about 290 m²/g, about 300 m²/g, about 310 m²/g, about 320 m²/g, about 330 m²/g, about 340 m²/g, about 350 m²/g, about 360 m²/g, about 370 m²/g, about 380 m²/g, about 390 m²/g, about 400 m²/g, about 410 m²/g, about 420 m²/g, about 430 m²/g, about 440 m²/g, about 450 m²/g, about 460 m²/g, about 470 m²/g, about 480 m²/g, about 490 m²/g, about 500 m²/g, or within any range defined by these values (e.g., from about 390 m²/g to about 420 m²/g).

In at least one embodiment, a pore volume of the zeolite is about 0.20 cm³/g, about 0.21 cm³/g, about 0.22 cm³/g, about 0.23 cm³/g, about 0.24 cm³/g, about 0.25 cm³/g, about 0.26 cm³/g, about 0.27 cm³/g, about 0.28 cm³/g, about 0.29 cm³/g, about 0.30 cm³/g, about 0.31 cm³/g, about 0.32 cm³/g, about 0.33 cm³/g, about 0.34 cm³/g, about 0.35 cm³/g, about 0.36 cm³/g, about 0.37 cm³/g, about 0.38 cm³/g, about 0.39 cm³/g, about 0.40 cm³/g, or within any range defined by these values (e.g., from about 0.26 cm³/g to about 0.31 cm³/g). As used herein, “pore volume” refers to pore volume as measured using N₂ adsorption-desorption and computed according to the Barrett, Joyner, and Halenda (BJH) method or mercury (Hg) porosimetry.

In at least one embodiment, the amount of copper dispersed onto/into the zeolite is about 2 mol %, about 2.25 mol %, about 2.5 mol %, about 2.75 mol %, about 3 mol %, about 3.25 mol %, about 3.5 mol %, about 3.75 mol %, about 4 mol %, about 4.25 mol %, about 4.5 mol %, about 4.75 mol %, about 5 mol %, about 5.25 mol %, about 5.5 mol %, about 5.75 mol %, about 6 mol %, about 6.25 mol %, about 6.5 mol %, about 6.75 mol %, about 7 mol %, about 7.25 mol %, about 7.5 mol %, about 7.75 mol %, about 8 mol %, about 8.25 mol %, about 8.5 mol %, about 8.75 mol %, about 9 mol %, about 9.25 mol %, about 9.5 mol %, about 9.75 mol %, about 10 mol %, or within any range defined by these values (e.g., from about 3 mol % to about 6 mol %) based on a total amount of all components of the copper-impregnated zeolite.

The zeolite may also be characterized in terms of oxidizable sites. In at least one embodiment, an amount of oxidizable sites by N₂₀ of the zeolite is about 10 μmol/g, about 11 μmol/g, about 12 μmol/g, about 13 μmol/g, about 14 μmol/g, about 15 μmol/g, about 16 μmol/g, about 17 μmol/g, about 18 μmol/g, about 19 μmol/g, about 20 μmol/g, about 21 μmol/g, about 22 μmol/g, about 23 μmol/g, about 24 μmol/g, about 25 μmol/g, about 26 μmol/g, about 27 μmol/g, about 28 μmol/g, about 29 μmol/g, about 30 μmol/g, or within any range defined by these values (e.g., from about 13 μmol/g to about 22 μmol/g).

In at least one embodiment, the catalyst is in the form of particles, which may be admixed with other types of catalytic particles. In at least one embodiment, the catalyst is in the form of an extrudate.

### Methods of Use

Certain embodiments relate to a reactor system utilizing the aforementioned catalysts to convert a syngas feed into a product stream comprising branched alkanes. The embodiments may be utilized advantageously by an offshore platform where the resulting product stream can be fed directly into a crude stream of a crude oil pipeline. For example, a H₂/CO ratio of the syngas feed is from about 1.5 to about 3 (e.g., about 1.5 to about 2.7).

In at least one embodiment, the method utilizes a first reactor stage comprising a first catalyst bed housed within a first reaction vessel, where the catalyst bed is made up of catalyst particles/granules. In at least one embodiment, the first catalyst bed comprises particles/granules of a first catalyst adapted for at least partial conversion of syngas into DME and methanol, resulting in a treated stream comprising DME, methanol, and unconverted hydrogen from the syngas feed.

In at least one embodiment, the method further a second catalyst bed housed within a second reaction vessel. In at least one embodiment, the second catalyst bed comprises a mixture of the first catalyst and a second catalyst, with the second catalyst comprising a HOG catalyst as described above.

In at least one embodiment, the method further utilizes a second reactor stage comprising a third catalyst bed of solely the second catalyst without the presence of the first catalyst. The product stream leaving the third catalyst bed may comprise branched alkanes, such as triptane, and minimal DME and methanol (e.g., less than 0.5 wt % methanol). In at least one embodiment, the reaction conditions of one or more of the first stage or the second stage comprise a temperature of about 250° C. to about 280° C. and a pressure of about 10 bar to about 40 bar.

Without wishing to be bound by theory, it is believed that the mixed catalysts of the second catalyst bed can advantageously push the methanol/DME equilibrium further towards DME to facilitate further conversion of DME, and thereafter conversion to a high-octane gasoline product stream by the HOG catalyst. This result is evidenced by the minimal methanol observed at the HOG catalyst stage at a temperature of greater than 260° C.

In at least one embodiment, the HOG catalyst is pretreated with a paraffin/olefin pretreatment stream at about 200° C. for improved performance. For example, the paraffin/olefin pretreatment stream comprises at least about 0.5 mol % propane in propylene. In at least one embodiment, the paraffin/olefin pretreatment stream comprises C3 and C4 olefins.

In at least one embodiment, the HOG catalyst is conditioned by operating the maximum temperature of the bed between about 275° C. and 320° C. under a flowing mixture of 1:1 molar ratio DME and hydrogen before the catalyst bed is brought back to steady state conditions.

In at least one embodiment, the first catalyst bed and the second catalyst bed are housed in the same reactor vessel, with the second catalyst bed being downstream from the first catalyst bed.

In at least one embodiment, the two-stage reactor is in a parallel fixed bed configuration, for example, when catalyst active lifetimes are greater than about 100 hours and where the second stage has a parallel bed to be regenerated while offline. In at least another embodiment, the two-stage reactor is in a moving bed configuration, for example, when catalyst active lifetimes are less than about 100 hours.

In at least one embodiment, the conversion of the syngas feed into the product stream is performed via one pass in a single-pass reactor.

In at least one embodiment, the product stream contains less than about 4 wt %, less than about 2 wt %, or less than about 1 wt % hexamethylbenzene.

In at least one embodiment, the product stream is separated into a predominantly aqueous stream and a predominantly organic stream. The organic stream may include light fractions solubilized in reaction products. The organic stream may have an octane rating of greater than 90 or greater than 95, and can be introduced directly into a crude oil pipeline.

In at least one embodiment, the product stream is first cooled to a temperature from about 30° C. to about 70° C. Remaining organic vapor can be utilized for steam generation and/or for feeding into an upstream flash vessel of a crude oil stream.

The following exemplary embodiments are now described:

Embodiment 1: A method for converting an offshore syngas feed into a product stream comprising branched alkanes, the method comprising: directing the syngas feed to a first catalyst bed comprising a first catalyst to produce a treated stream, the treated stream comprising dimethyl ether (DME), methanol, and unconverted hydrogen generated from contact of the syngas feed with the first catalyst bed; directing the treated stream toward a second catalyst bed comprising a mixture of the first catalyst and a second catalyst; and subsequently directing the treated stream toward a third catalyst bed comprising the second catalyst without the first catalyst to at least partially convert the treated stream into the product stream.

Embodiment 2: The method of Embodiment 1, wherein the conversion of the syngas feed into the product stream is performed via one pass in a single-pass reactor.

Embodiment 3: The method of either Embodiment 1 or Embodiment 2, further comprising: separating the product stream into a predominantly aqueous stream and a predominantly organic stream comprising light fractions solubilized in reaction products; and introducing the organic stream into a crude oil pipeline.

Embodiment 4: The method of Embodiment 3, wherein the organic stream has a Research Octane Number (RON) of greater than 90 or greater than 95.

Embodiment 5: The method of Embodiment 3, wherein the organic stream has greater than 5 wt % of aliphatic hydrocarbons with a RON of greater than 100.

Embodiment 6: The method of any one of the preceding Embodiments, wherein the syngas feed is contacted with the first catalyst bed in a first reactor of a two-stage reactor, and wherein the treated stream is contacted with the second catalyst bed and the third catalyst bed in a second reactor of the two-stage reactor.

Embodiment 7: The method of Embodiment 6, wherein the reaction conditions of one or more of the first reactor or the second reactor comprise a temperature of about 250° C. to about 280° C., or about 255° C. to about 265° C., and a pressure of about 10 bar to about 40 bar, or about 15 bar to about 20 bar.

Embodiment 8: The method of Embodiment 7, wherein the reaction conditions further comprise a flowrate of about 0.3 to about 15 g_{DME}/g_{catalyst}/hour, or about 0.5 to about 1.5 g_{DME}/g_{catalyst}/hour.

Embodiment 9: The method of Embodiment 6, wherein the two-stage reactor is in a parallel fixed bed configuration.

Embodiment 10: The method of Embodiment 6, wherein the two-stage reactor is in a moving bed configuration.

Embodiment 11: The method of any one of the preceding Embodiments, wherein contact of the treated stream with the second catalyst bed further converts additional methanol to DME.

Embodiment 12: The method of any one of the preceding Embodiments, wherein a H₂/CO ratio of the syngas feed is from about 1.5 to about 3, and a CO₂ concentration of the syngas feed is from about 5 to about 10 mol %.

Embodiment 13: The method of any one of the preceding Embodiments, wherein the first catalyst comprises ZnO, CuO, alumina, and optionally ZrO₂.

Embodiment 14: The method of any one of the preceding Embodiments, wherein the first catalyst further comprises one or more of boron oxide, niobium oxide, tantalum oxide, phosphorus oxide, or combinations thereof.

Embodiment 15: The method of either Embodiment 13 or Embodiment 14, wherein the first catalyst further comprises ceria.

Embodiment 16: The method of any one of the preceding Embodiments, wherein the second catalyst comprises a zeolite.

Embodiment 17: The method of Embodiment 16, wherein the zeolite is a copper-impregnated beta zeolite.

Embodiment 18: The method of Embodiment 17, wherein the copper-impregnated beta zeolite comprises copper from about 2 wt. % to about 10 wt. % based on a total weight of the copper-impregnated zeolite.

Embodiment 19: The method of either Embodiment 17 or Embodiment 18, wherein the copper-impregnated zeolite further comprises one or more additional metals from about 0.5 wt % to about 3 wt %, wherein the one or more additional metals are selected from metals having an H₂ adsorption energy of 77 KJ/mol to 124 KJ/mol on (111), (110), or (0001) surfaces.

Embodiment 20: The method of any one of Embodiments 16-18, wherein a silica-to-alumina ratio (SAR) of the zeolite is from about 25 to about 100, or from about 30 to about 60.

Embodiment 21: The method of Embodiment 17, wherein a total surface area of the copper-impregnated beta zeolite is from about 350 m²/g to about 450 m²/g, and wherein a pore volume of the copper-impregnated beta zeolite is from about 0.2 cm³/g to about 0.4 cm³/g.

Embodiment 22: The method of Embodiment 17, wherein an amount of sites oxidizable by N₂O on the copper-impregnated beta zeolite is from about 10 μmol/g to about 30 μmol/g.

Embodiment 23: The method of any one of the preceding Embodiments, wherein conversion of the treated stream into the product stream results in less than about 4 wt % hexamethylbenzene.

Embodiment 24: The method of any one of the preceding Embodiments, further comprising: cooling the product stream to a temperature from about 30° C. to about 70° C.; and utilizing remaining organic vapor for steam generation and/or feeding the remaining organic vapor to a vessel where the vapor is contacted with a liquid crude oil stream at ambient pressure and at a flow rate that is at least 5 times higher than the flow rate of the product stream.

Embodiment 25: The method of Embodiment 1, wherein a methanol content of the product stream is less than about 0.5 wt %, or less than about 0.05 wt %.

Embodiment 26: The method of any one of the preceding Embodiments, further comprising: pretreating the second catalyst with a paraffin/olefin pretreatment stream at about 200° C.

Embodiment 27: The method of Embodiment 26, wherein the paraffin/olefin pretreatment stream comprises at least about 0.5 mol % propane in propylene.

Embodiment 28: The method of Embodiment 26, wherein the paraffin/olefin pretreatment stream comprises C3 and C4 olefins.

Embodiment 29: A system for converting a syngas feed into a product stream comprising branched alkanes, the system comprising: a first reactor adapted to receive a syngas feed stream, the first reactor comprising a first catalyst adapted to produce a treated stream comprising dimethyl ether (DME) and methanol generated from contact with the syngas feed stream; and a second reactor downstream from the first reactor adapted to receive the treated stream, the second reactor comprising a first bed of a mixture of a first and second catalyst and a second bed comprising a second catalyst adapted to at least convert the treated stream into the product stream.

### ILLUSTRATIVE EXAMPLES

The following examples are set forth to assist in understanding the disclosure and should not be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### Experimental Setup

Experiments were completed using a 300 cubic centimeter PARR® tubular reactor having 1″ diameter. An oil jacket was used for heating the catalyst bed to the desired temperature. The entire catalyst bed was approximately 20 cm long. 67 g of extruded catalyst (2 mm Ø×5 mm length) was used. This amount was used so that the top of the bed would be 9″ below the start of the oil jacket and the bottom of the bed would be 7″ above the bottom of the oil jacket. Process gas entered the insulated jacket at 180° C. and quickly reached reaction temperatures due to the coil in the upper third heating section. Two types of extruded beta zeolite catalysts were used having the characteristics described in in Table 1. The catalyst loaded only with copper is referred to herein as “Cu BEA” while the catalyst loaded with copper and nickel is referred to herein as “Cu/Ni BEA”. Copper loading was performed via aqueous ion exchange with Cu(NO₃)₂ solution.

TABLE 1
Characteristics of the catalysts tested

| | | | | BET | Pore | Pore | Cu | N₂O |
|---|---|---|---|---|---|---|---|---|
| | | | | Area | Diameter | Volume | Dispersion | Dissociated |
| | SAR | Cu | Ni | (m²/g) | (nm) | (cm³/g) | (mol %) | (μmol/g) |
| Cu BEA | 47.5 | 4.8% | — | 410 | 14.4^{a} | 0.287^{a} | 4.5 | 16.9 |
| | | | | | 8.2^{b} | 0.264^{b} | | |
| Cu/Ni | 47.5 | 4.7% | 1.2% | 403 | 14.7^{a} | 0.284^{a} | 5.4 | 20.0 |
| BEA | | | | | 7.91^{b} | 0.240^{b} | | |

^{a}Hg porosimetry
^{b}BJH desorption

DME was fed in as a compressed liquid through a subcooled HPLC pump, then heated to a vapor at temperatures >145° C. Pure hydrogen was mixed with the DME vapor and the mixture was maintained above 80° C. Heat trace operating at 180° C. was placed around the ¼″ tubing at the reactor outlet to maintain hexamethylbenzene (HMB) in the liquid and vapor phases. A knockout pot was operated at 167° C. to remove liquid HMB. A slip stream from the resulting vapor stream was fed to a GC inlet heated at 150° C. while the rest was cooled and fed to a series of filters in ¾″ tubing to collect any HMB undergoing deposition at ambient temperature. Once cooled to ambient temperature, the high-pressure liquid was separated from the non-condensables. Hydrogen and propylene feed stream mass flow controllers had totalizers built in to record the amount of mass in. A steady feed of DME was maintained through the pump, but due to the nature of the totalizer on the HPLC pump, the compressed DME feed rate had to be calculated based off the vol % of DME after mixed with hydrogen.

### Catalyst Activation

The catalyst was activated by flowing pure hydrogen at 13 mL H₂/g_{Catalyst}/min for the first hour followed by 5 mLH₂/g_{Catalyst}/min for the subsequent hour(s) while the catalyst bed was maintained at 290° C. at 5-10 psig. After this step, the catalyst was held at 210° C. under 50 standard cubic centimeters per minute (SCCM) of flowing nitrogen overnight. Any time the catalyst was heated, the temperature was not increased more than 2.5° C./min. Catalyst conditioning was performed after the initial hydrogen reduction. This involved flowing a 99% propylene/1% propane mixture flowing at 300 SCCM at 210° C. and 5-10 psig for 10 minutes.

### DME Conversion Experiments

For the offshore application, single pass conversion is advantageous for eliminating the need for additional compression of a recycle stream. The focus for the reaction was at temperatures greater than 240° C., but the catalyst bed did not exceed temperatures of 320° C. Process parameter ranges are listed in Table 2.

TABLE 2
Range of reaction conditions investigated

| Parameter | Min | Max |
|---|---|---|
| Temperature (° C.) | 210 | 280 |
| Reactor Pressure (psig) | 50 | 220 |
| WHSV (g_{DME}/g_{Catalyst}/hour) | 0.3 | 1.1 |

Liquid samples were collected from the gas liquid separator and from the HMB knockout pot and vapor reactor product samples were collected from the vapor outlet of the HMB knockout pot. Gas samples were analyzed on a Q-BOND column via flame ionization detection (FID) gas chromatography (GC), and a molecular sieve column via thermal conductivity (TCD) GC. The GC method began with an oven temperature at 40° C. and increased to 240° C. at 20° C./min. Standards for the FID and TCD methods are shown in Table 3. Liquid standards were injected via the septum valve on the FID loop. These general hydrocarbons ensured proper response times and response factors.

TABLE 3
Composition of GC standards by weight

| | | Liquid | |
|---|---|---|---|
| Component | Liquid Standard | Standard #2 | Vapor Standard |
| Hydrogen | | | 2% |
| Methane | | | 2% |
| Propane | | | 4% |
| i-Butane | | | 40% |
| Dimethyl ether | | | 10% |
| Nitrogen | | | 17% |
| 2-methylbutane | 0.4% | 3% | 25% |
| Pentane | 0.4% | 3% | |
| Hexane | 1.7% | | |
| Triptane | 2.6% | 19% | |
| Octane | 4.9% | 36% | |
| Methanol | 90% | | |
| 2,3-dimethylpentane | | 3% | |
| 2-methylhexane | | 5% | |
| 2,2-dimethylbutane | | 22% | |
| 2,3-dimethylbutane | | 10% | |

### Results

The results of the DME conversion experiments described above are summarized below in Table 4. Conversion was determined by calculating the total amount of carbon in the outlet stream compared to the number of DME carbons still present. Carbon selectivity was determined from the total number of carbon in the hydrocarbon products in the reactor effluent.

TABLE 4
Summary of experimental results

| | Reactor | Avg Catalyst | WHSV | Feed | | | | |
|---|---|---|---|---|---|---|---|---|
| | Pressure | Temperature | (g_{DME}/ | Ratio | | Methanol | Triptane | C₄+ |
| Catalyst | (psig) | (° C.) | g_{Catalyst}/h) | H₂:DME | X_{DME} | (wt %) | C-selectivity | C-selectivity |
| Cu/Ni BEA | 50 | 258 | 0.31 | 0.7:1 | 99.7% | 0.9% | 13.1% | 83% |
| Cu/Ni BEA | 220 | 264 | 0.98 | 0.9:1 | 99.9% | 0.0% | 9.4% | 75% |
| Cu/Ni BEA | 220 | 260 | 0.34 | 1.1:1 | 99.8% | 0.2% | 10.6% | 83% |
| Cu BEA | 220 | 260 | 0.37 | 1.1:1 | 99.96% | 0.3% | 12.4% | 84% |
| Cu BEA | 220 | 274 | 1.1 | 1.1:1 | 99.8% | 0.0% | 13.5% | 83% |
| Cu BEA | 220 | 275 | 1.1 | 1.1:1 | 99.8% | 0.4% | 14.0% | 84% |
| Cu BEA | 220 | 256 | 1.1 | 1.1:1 | 99.9% | 1.0% | 12.9% | 80% |
| Cu BEA | 50 | 249.4 | 0.34 | 1.1:1 | 98.2% | 0.0% | 16.0% | 90% |
| Cu BEA | 100 | 249.2 | 0.34 | 1.1:1 | 99.4% | 0.4% | 15.7% | 89% |
| Cu BEA | 150 | 249.1 | 0.38 | 1.1:1 | 99.9% | 0.2% | 12.6% | 86% |
| Cu BEA | 220 | 251.4 | 0.37 | 1.1:1 | 83.7% | 7.3% | 16.0% | 85% |

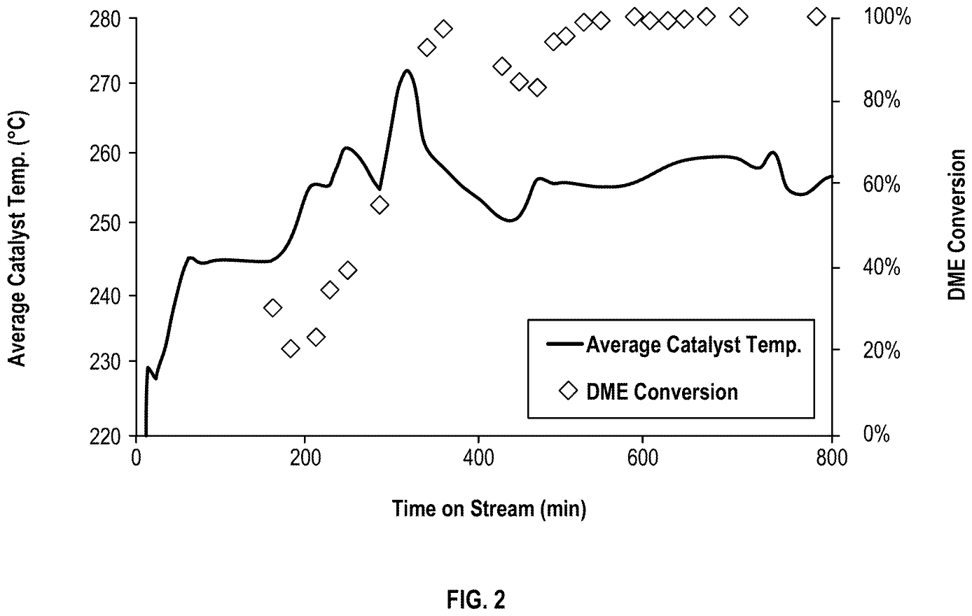

The catalyst exhibited a series of two exotherms when first exposed to the reactant mixture, the second of which points to hysteresis behavior for these catalysts. An exotherm of <15° C. was seen when the DME/hydrogen mixture first reached the catalyst bed while the oil jacket temperature was at 240° C. for 220 psig start-ups and 220° C. for 50 psig start-ups. As the oil jacket temperature was increased, DME conversion would reach 40-60%, at which point the catalyst bed temperature would start to steadily increase. While at about 0.35 weight hourly space velocity (WHSV), the maximum temperature would reach 270° C. to 290° C. before it would peak. While at about 1.1 WHSV, the maximum temperature would reach 300° C. to 320° C. before it would peak. Once the catalyst temperature reached roughly 280° C., the oil jacket temperature would start to be decreased by the operator. As shown in FIG. 2, after this larger exotherm at high conversion, less extreme temperatures would be needed to maintain higher conversion. Specifically at an average catalyst temperature of 256° C., before this exotherm at 230 minutes TOS, the DME conversion was only 35%. At that time, the entire catalyst bed was within 2.5° C., ranging from 254.5° C. at the bottom to 257° C. at the top. By 550 minutes TOS after the exotherm at this temperature, DME conversion was >98%. The higher conversion was observed by the changes in the catalyst bed temperature profile; the top of the bed measured 266° C., the middle of the bed measured 258° C., and the bottom of the bed measured 249.6° C. Due to this phenomenon, the most representative samples were assumed to occur after this initial higher exotherm subsided. This post exotherm behavior was also beneficial for stable control. After this exotherm, the catalyst bed could be operated at temperatures that generated 90-100% DME conversion without causing the bed temperature to run away.

The Cu BEA catalyst that was prepared with a copper (II) acetate solution in previous experiments exhibited a short lifetime. Within an hour of TOS, the DME conversion would decrease to <10%. However, both catalysts prepared as described above operated very well over long run times. The Cu/Ni BEA catalyst operated for >75 hours TOS without showing signs of deactivation. The Cu BEA catalyst operated for >50 hours TOS without showing signs of deactivation. Both catalysts were held under 50 SCCM of flowing hydrogen overnight at temperatures of 210-230° C. after having been exposed to hydrogen/DME fixtures for up to 16 hours continuously each day. It was observed that at low pressure (50 psig) the catalyst would not have to reach temperatures >270° C. to reach high conversion again after this flowing hydrogen idle state. However, when the catalyst was exposed to flowing hydrogen at 220 psig overnight, the catalyst would have to reach these high temperatures again to exhibit this increased activity at lower temperatures.

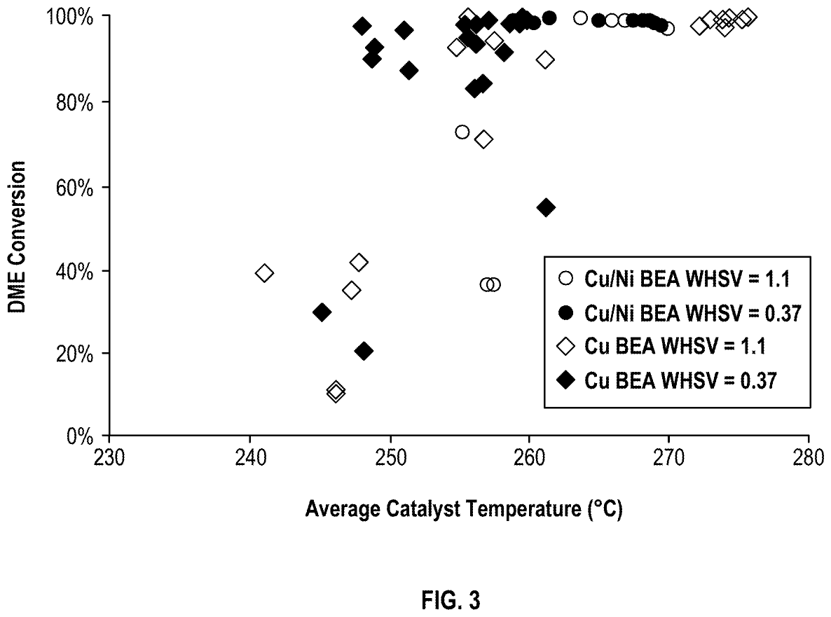

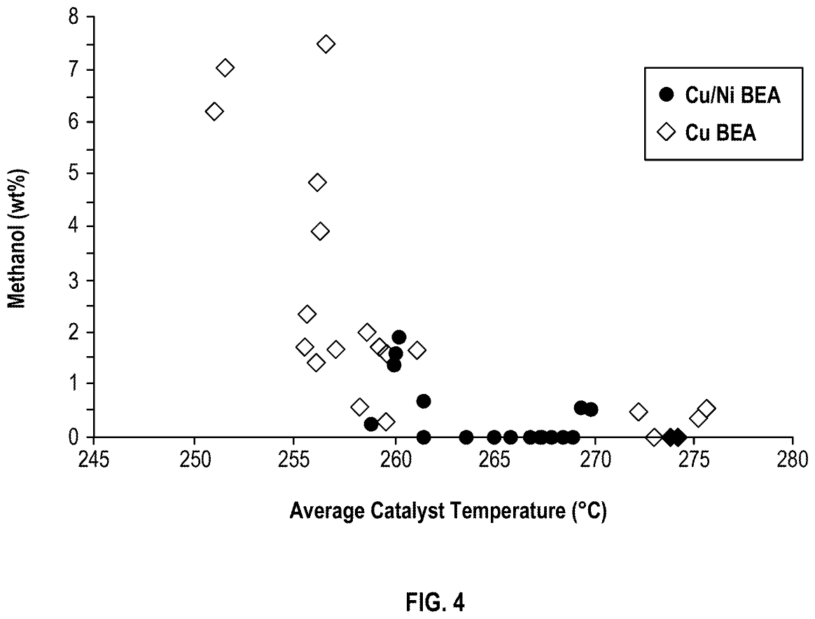

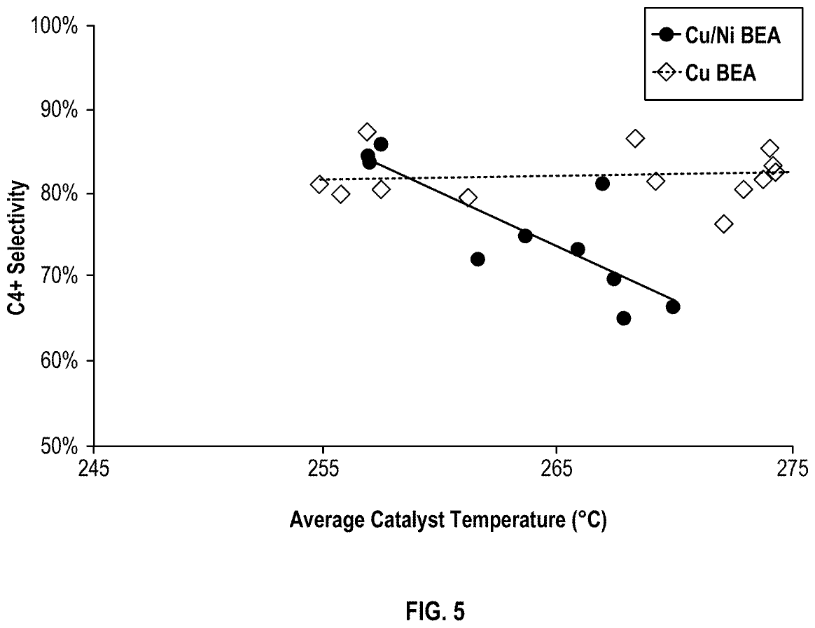

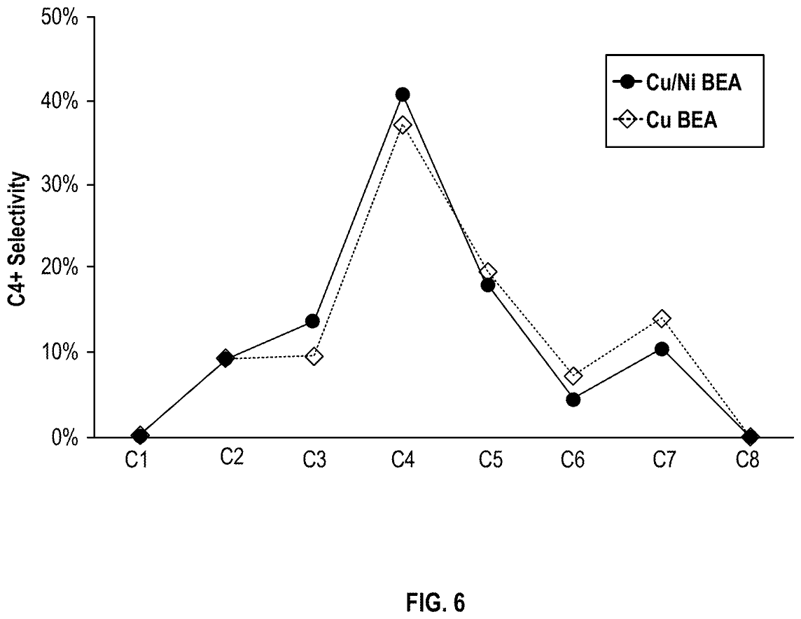

As the WHSV was increased from 0.37 to 1.1, it was observed that >99% conversion occurred at higher temperatures. As shown in FIG. 3, the high flowrate data reaches >99% DME conversion 6-8° C. higher than the lower flowrates for both catalysts. There was no further data collected at feed rates higher than 1.1 WHSV, but higher flowrates are possible. For example, with the Cu BEA catalyst, >99% conversion was achieved at 256° C. At higher WHSV, higher reactor temperature may be advantageous, but there is ample operational flexibility to increase temperature. This increase in temperature is beneficial for the reduction in methanol as shown in FIG. 4 (where WHSV varied between 0.37 and 1.1, pressure was at 220 psig, and water and non-condensables were included in the wt % calculation). Below an average catalyst temperature of about 262° C., the methanol in the product stream is >0.5 wt %. High amounts of methanol would accumulate in the aqueous phase separation, which would not be advantageous to the offshore process. The hydration of DME to methanol is so prevalent due to the high amount of water generated, therefore, the levels of methanol remain high until almost all the DME is consumed. Any presence of DME at relatively high conversion resulting in high water content pushes the equilibrium in favor of methanol. Controlling the amount of methanol in the reactor effluent would not be difficult because the methanol sharply drops off above 260° C., or X_{DME}>99.8%, and can be reduced to 0 at high enough temperatures. Increasing the temperature substantially with the Cu BEA catalyst is feasible, however, as observed in FIG. 5, C4+C-selectivity decreases with increasing temperature for the Cu/Ni BEA catalyst (under conditions of 1.1 WHSV and 220 psig reactor pressure). The performance of the two catalysts was similar <260° C., but only the Cu BEA catalyst maintained the high C4+C-selectivity at higher temperatures. At high conversion, both catalysts show similar C-selectivity, as observed in FIG. 6 (for 99.9% DME conversion at 220 psig reactor pressure). The Cu BEA shows a slight improvement in the liquid fraction, generating 84% C4+C-selectivity and 14% triptane C-selectivity. This triptane C-selectivity is significantly greater than the 9.8% triptane C-selectivity observable with the Cu/Ni BEA at high feed rates and high conversion. This can be advantageous in increasing the overall octane number of the gasoline fraction of the crude oil stream when using the Cu BEA catalyst. Both catalysts showed very high activity since no ethylene or propylene was detected in the reactor effluent, suggesting that olefinic compounds were immediately converted to higher alkanes. Table 5 shows a simulated product composition based on experimental results at 275° C., 220 psig, and 1.1 WHSV when feeding 1.1:1 H₂:DME over a Cu BEA catalyst bed (X_{DME}=99.8%).

TABLE 5
Simulated product composition

| Component | Mass % |
|---|---|
| Methane | 0.7% |
| Ethane | 4.6% |
| Propane | 2.5% |
| Isobutane | 20.1% |
| Butane | 1.9% |
| Isopentane | 10.5% |
| Pentane | 0.7% |
| 2,2 dimethylbutane | 2.8% |
| 2,3 dimethylbutane | 2.9% |
| Benzene | 0.3% |
| Unknown C₆ compounds | 0.3% |
| Triptane | 8.6% |
| 2,3 dimethylpentane | 0.5% |
| 2-methylhexane | 0.4% |
| 2,2,4-trimethylpentane | 1.5% |
| 2,2,4-trimethylpentene | 0.7% |
| Hydrogen | 3.4% |
| Water | 35.6% |
| DME | 0.2% |
| Methanol | 0.4% |
| Hexamethylbenzene | 1.6% |

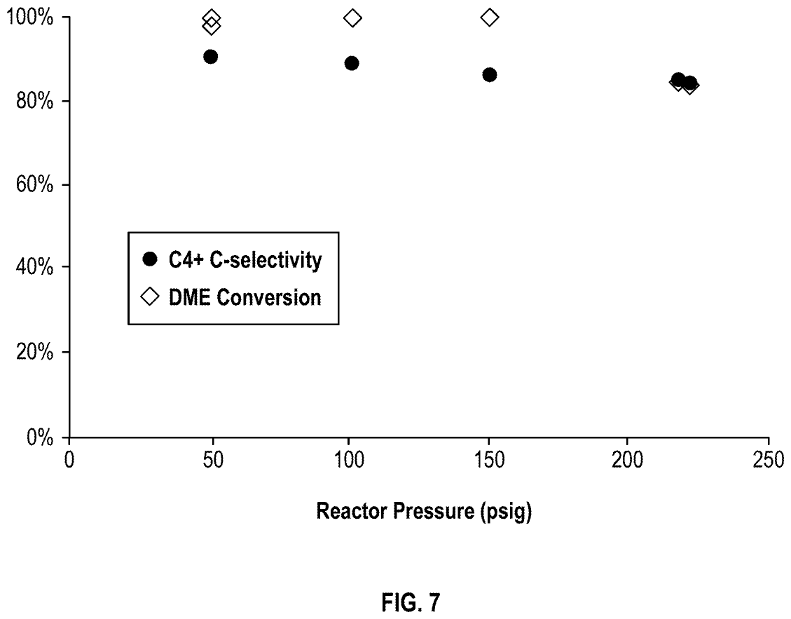

As reactor pressure is increased from 50 psig to 150 psig, DME conversion holds steady near 100% while the C4+C-selectivity decreases by 4%. As shown in FIG. 7, between 150 psig and 220 psig there is a significant drop off in conversion while the average reactor temperature is maintained at 250° C. (for the Cu BEA catalyst at 250° C. and 0.37 WHSV). If pressures >150 psig negatively affected the DME conversion, higher temperature might only moderately improve the conversion. However, it is observed that with a small increase in temperature up to 255° C., DME conversion once again reaches >99% while still at 220 psig. This fast conversion response to temperature suggests that HMB formed at these conditions is condensing on the catalyst surface. Once the temperatures increase, HMB remains in the vapor phase and the catalyst activity resumes to levels observable at lower pressures.

HMB could not be removed as a liquid at lower pressures since it goes through a deposition phase change at the low partial pressure present in the reactor effluent. As the pressure was increased to 220 psig, a liquid mixture with HMB could be removed from the knockout pot at 167° C. However, HMB still built up in the ambient temperature filters downstream of the knockout pot vapor outlet. Simulations with Aspen Plus® estimate that this liquid collected to remove about 80% of the HMB produced would be roughly 43 wt % water. This would lead to difficult collection and removal of HMB. Therefore, it would be advantageous to selectively remove solid, crystalline HMB in a parallel exchanger set-up during the downstream vapor/liquid separation in a commercial process. These exchangers could be quickly cleaned out by dissolving the HMB in high pressure crude oil. While both catalysts produced HMB, the Cu/Ni BEA catalyst yielded roughly 30% less HMB than the Cu BEA catalyst, likely due to the higher hydrogenation characteristics of the Ni. Both catalysts had HMB yields between 1 and 2%. The Cu/Ni BEA catalyst would be expected to have higher hydrogenation activity, and its C3 C-selectivity was 4% higher than the Cu BEA catalyst (see FIG. 6). With these points, there appears to be a tradeoff between the C4+ fraction and HMB yield.

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment.

The present disclosure has been described with reference to specific exemplary embodiments thereof. The specification and drawings are, accordingly, to be regarded in an illustrative rather than a restrictive sense. Various modifications of the disclosure in addition to those shown and described herein will become apparent to those skilled in the art and are intended to fall within the scope of the appended claims.

1. A method for converting an offshore syngas feed into a product stream comprising branched alkanes, the method comprising:
directing the syngas feed to a first catalyst bed comprising a first catalyst to produce a treated stream, the treated stream comprising dimethyl ether (DME), methanol, and unconverted hydrogen generated from contact of the syngas feed with the first catalyst bed;
directing the treated stream toward a second catalyst bed comprising a mixture of the first catalyst and a second catalyst; and
subsequently directing the treated stream toward a third catalyst bed comprising the second catalyst without the first catalyst to at least partially convert the treated stream into the product stream.

2. The method of claim 1, wherein the conversion of the syngas feed into the product stream is performed via one pass in a single-pass reactor.

3. The method of claim 1, further comprising:
separating the product stream into a predominantly aqueous stream and a predominantly organic stream comprising light fractions solubilized in reaction products; and
introducing the organic stream into a crude oil pipeline.

4. The method of claim 3, wherein the organic stream has a Research Octane Number (RON) of greater than 90 or greater than 95.

5. The method of claim 3, wherein the organic stream has greater than 5 wt % of aliphatic hydrocarbons with a RON of greater than 100.

6. The method of claim 1, wherein the syngas feed is contacted with the first catalyst bed in a first reactor of a two-stage reactor, and wherein the treated stream is contacted with the second catalyst bed and the third catalyst bed in a second reactor of the two-stage reactor.

7. The method of claim 6, wherein the reaction conditions of one or more of the first reactor or the second reactor comprise a temperature of about 250° C. to about 280° C., or about 255° C. to about 265° C., and a pressure of about 10 bar to about 40 bar, or about 15 bar to about 20 bar.

8. The method of claim 7, wherein the reaction conditions further comprise a flowrate of about 0.3 to about 15 g_{DME}/g_{catalyst}/hour, or about 0.5 to about 1.5 g_{DME}/g_{catalyst}/hour.

9. The method of claim 6, wherein the two-stage reactor is in a parallel fixed bed configuration.

10. The method of claim 6, wherein the two-stage reactor is in a moving bed configuration.

11. The method of claim 1, wherein contact of the treated stream with the second catalyst bed further converts additional methanol to DME.

12. The method of claim 1, wherein a H₂/CO ratio of the syngas feed is from about 1.5 to about 3, and a CO₂ concentration of the syngas feed is from about 5 to about 10 mol %.

13. The method of claim 1, wherein the first catalyst comprises ZnO, CuO, alumina, and optionally ZrO₂.

14. The method of claim 1, wherein the first catalyst further comprises one or more of boron oxide, niobium oxide, tantalum oxide, phosphorus oxide, or combinations thereof.

15. (canceled)

16. The method of claim 1, wherein the second catalyst comprises a copper-impregnated beta zeolite.

17-20. (canceled)

21. The method of claim 16, wherein a total surface area of the copper-impregnated beta zeolite is from about 350 m²/g to about 450 m²/g, and wherein a pore volume of the copper-impregnated beta zeolite is from about 0.2 cm³/g to about 0.4 cm³/g.

22. The method of claim 16, wherein an amount of sites oxidizable by N₂O on the copper-impregnated beta zeolite is from about 10 μmol/g to about 30 μmol/g.

23. The method of claim 1, wherein conversion of the treated stream into the product stream results in less than about 4 wt % hexamethylbenzene.

24. The method of claim 1, further comprising:
cooling the product stream to a temperature from about 30° C. to about 70° C.; and
utilizing remaining organic vapor for steam generation and/or feeding the remaining organic vapor to a vessel where the vapor is contacted with a liquid crude oil stream at ambient pressure and at a flow rate that is at least 5 times higher than the flow rate of the product stream.

25-28. (canceled)

29. A system for converting a syngas feed into a product stream comprising branched alkanes, the system comprising:
a first reactor adapted to receive a syngas feed stream, the first reactor comprising a first catalyst adapted to produce a treated stream comprising dimethyl ether (DME) and methanol generated from contact with the syngas feed stream; and
a second reactor downstream from the first reactor adapted to receive the treated stream, the second reactor comprising a first bed of a mixture of a first and second catalyst and a second bed comprising a second catalyst adapted to at least convert the treated stream into the product stream.
